(19) United States
(12) Patent Application Publication
(10) Pub. No.: US 2026/0263259 A1
(43) Pub. Date: Sep. 10, 2026

(54) Menstrual Cup and Disc Applicator with Insertion and Removal Functions

(71) Applicant: LEVI PARKER, Long Beach, CA (US)

(72) Inventor: LEVI PARKER, Long Beach, CA (US)

(21) Appl. No.: 19/071,474

(22) Filed: Mar. 5, 2025

(51) Int. Cl. A61F 5/44; A61F 5/455

(52) U.S. Cl. CPC A61F 5/4404; A61F 5/4553

(57) ABSTRACT

The present invention relates to a multifunctional menstrual product applicator system designed to simplify the insertion, removal, and maintenance of menstrual cups and discs. The system includes a menstrual cup applicator with a notched end for securely holding and releasing folded menstrual cups and a disc inserter/remover rod with dual functionality—one end designed to hold and insert folded menstrual discs and the opposite end featuring a hook for removing discs. Both applicators feature ergonomic handles for user comfort and precision. Constructed from medical-grade materials, the system ensures durability, safety, and compatibility with sterilization processes. By offering customizable insertion depth, compatibility with various product sizes, and reusable components, the invention promotes sustainability, accessibility, and an improved user experience.

### FIELD OF THE INVENTION

The present invention relates to menstrual hygiene devices and, more particularly, to an applicator system for the insertion and removal of menstrual cups and menstrual discs. The system is designed to improve ease of use, hygiene, and comfort for users of reusable menstrual products.

### BACKGROUND OF THE INVENTION

The use of menstrual cups and menstrual discs as sustainable alternatives to traditional disposable sanitary products has grown significantly in recent years. Menstrual cups and discs are reusable, eco-friendly, cost-effective, and provide longer wear time compared to pads and tampons. These products are typically made from medical-grade silicone, rubber, or thermoplastic elastomer (TPE), and they function by collecting menstrual flow within the vaginal canal, rather than absorbing it.

Despite their benefits, menstrual cups and discs have not been widely adopted due to various challenges users face during insertion and removal. These challenges are primarily related to the difficulty of correctly folding, positioning, and ensuring a proper seal of the menstrual product within the vaginal canal. For many users, the insertion process can be uncomfortable and intimidating, particularly for individuals who are new to menstrual cups or those who have limited experience or dexterity. Removal can also be difficult, especially with menstrual discs, which tend to fit more snugly in the vaginal canal and are more challenging to retrieve without the proper tools.

The concept of a menstrual cup dates back to the early 20th century, but it wasn't until the 1980s that more commercially viable and widely available versions of the product were introduced. Early versions of menstrual cups were often made from rigid materials that were difficult to manipulate and insert. Over time, the design evolved to become more flexible and user-friendly, with modern menstrual cups being made from soft, medical-grade silicone that is comfortable and easily adapted to fit different body types.

While the benefits of menstrual cups—such as their reusability, cost-effectiveness, and environmentally friendly nature—are well documented, insertion remains one of the most common barriers for new users. In order to insert a menstrual cup, users typically need to fold it in one of several specific ways (such as the “C Fold,” “7 Fold,” or “Punch Down Fold”) before inserting it into the vaginal canal. For many individuals, mastering the proper folding technique and ensuring that the cup unfolds correctly inside the body can be challenging, particularly for those with less experience using these products or individuals with limited dexterity.

Additionally, once the menstrual cup is inserted, users must ensure that it has opened fully to create a secure seal around the cervix. Without proper positioning, the menstrual cup can leak, rendering it ineffective. This issue can discourage users from fully adopting menstrual cups, especially for those who may feel uncertain about their correct placement.

Menstrual discs are another innovative option in the market, offering a different form factor compared to menstrual cups. These discs are typically flatter and wider than menstrual cups, and they rest against the cervix, collecting menstrual fluid in a disc shape rather than the bell shape of a menstrual cup. Discs offer the potential for longer wear time, often up to 12 hours, and are discreet and comfortable once inserted correctly.

However, unlike menstrual cups, menstrual discs tend to be more difficult to remove because they do not have a stem or ring that can easily be grasped. Instead, users must rely on their fingers to pull the disc out, which can be difficult, especially when the disc is seated high inside the vaginal canal. To retrieve the disc, users must often push on the vaginal walls or use their fingers to hook and pull the disc out, which can be uncomfortable, particularly for individuals who are not accustomed to handling menstrual products internally.

As with menstrual cups, inserting a menstrual disc also requires skill. The disc must be folded before insertion, and users often struggle with inserting the disc into the correct position in the vaginal canal. Once inside, the disc must rest against the cervix and be securely positioned behind the pubic bone, which can be a difficult task without adequate guidance or tools.

Despite their significant advantages, menstrual cups and discs present unique challenges for users. These difficulties primarily stem from the complexity of the insertion and removal processes, which can be intimidating, uncomfortable, or inaccessible to some users. These challenges include:
Difficulty with Insertion: The process of folding and inserting a menstrual cup or disc requires precision and practice. Most users must fold the product into a specific shape before insertion, a process that requires careful manipulation and can be challenging for people with limited hand dexterity or first-time users. Even for experienced users, improper folding or insertion technique can result in discomfort or difficulty with achieving a secure seal. For menstrual cups, improper insertion or failure to unfold the cup fully inside the body can lead to leakage. Discs, while more flexible in shape, often have difficulty staying securely positioned without proper placement.
Difficulty with Removal: The removal of both menstrual cups and discs can be challenging. Menstrual cups have a stem that can be grasped for removal, but some individuals find it difficult to reach the stem or grasp it firmly enough to remove the cup. In particular, when a cup is positioned high in the vaginal canal, removal can be a daunting task without the right technique or tools. Menstrual discs present an even greater challenge because they lack an external stem or easy-to-reach grip. They sit further inside the vaginal canal, making them harder to grasp and remove. For some users, attempting to remove a disc can result in discomfort or frustration. Manual removal without guidance often leads to unsuccessful attempts, which can discourage users from continuing to use menstrual discs.
Hygiene and Comfort Concerns: When using menstrual products, hygiene and comfort are critical concerns. Traditional methods of inserting menstrual cups or discs often require significant manual handling, increasing the likelihood of bacterial contamination. Additionally, many users feel discomfort when attempting to insert or remove these products, particularly if they are unsure about how to properly position them or are concerned about incorrect placement.
Accessibility and Inexperience: For new users, menstrual cups and discs can feel intimidating due to a lack of understanding of the mechanics involved in inserting and removing the products. Without proper guidance or tools, individuals may feel hesitant to try reusable menstrual products. This results in a reliance on disposable products such as pads and tampons, which are less sustainable and less cost-effective over time.

The need for improved menstrual product applicators arises from the significant challenges users face during the insertion and removal of menstrual cups and discs, which can be uncomfortable, difficult, and intimidating, especially for new users or those with limited dexterity. Insertion often requires precise folding and positioning techniques, and incorrect placement can lead to leaks, discomfort, or improper sealing. Removal poses further challenges, particularly with menstrual discs, which lack an easy-to-grasp stem and can be positioned high in the vaginal canal, making retrieval difficult and frustrating. Additionally, traditional manual handling of these products increases the risk of contamination and compromises hygiene. An improved applicator system would simplify these processes, enhance user comfort, promote hygiene, and make reusable menstrual products more accessible, reducing the reliance on disposable alternatives and supporting sustainable menstrual care practices.

### SUMMARY OF THE INVENTION

The present invention provides an innovative multi-functional applicator system designed to simplify the insertion and removal of menstrual cups and menstrual discs, ensuring optimal comfort, hygiene, and proper placement for users. The invention specifically addresses the common challenges users face when using reusable menstrual products, such as difficulty with folding, insertion, and removal, as well as ensuring a proper seal and correct positioning.

In an embodiment of the present invention, the cup applicator assists in inserting a folded menstrual cup into the vaginal canal. It features a specially designed notched end to securely hold the cup and an insertion rod to gently release the cup into position. The applicator makes it easier for users to insert the menstrual cup without discomfort or fear of improper placement. The notched end ensures the cup stays folded during insertion, and the rod is used to push the cup into place, allowing it to unfold and form a secure seal around the cervix.

The disc inserter/remover rod is a dual-purpose tool designed to aid in both the insertion and removal of menstrual discs. It features a stable hook at one end, which can be used to retrieve the menstrual disc by catching its edge or tab. On the other end, the rod has notches that securely hold the folded menstrual disc during insertion. This tool ensures the menstrual disc is placed in the correct position within the vaginal canal, resting behind the pubic bone and securely covering the cervix to prevent leaks. Additionally, the removal process becomes more manageable, as users can insert the hooked end of the rod to pull the disc out gently.

The system includes a hygienic and compact storage holder designed to house and organize the applicator tools and lubricant. This holder keeps the tools clean and easily accessible while ensuring that they remain protected from contamination when not in use. It is made of durable, antimicrobial material to ensure cleanliness and ease of transportation, making it convenient for users on the go.

The invention offers a comprehensive solution to the challenges associated with using menstrual cups and discs. By providing an applicator system that simplifies insertion and removal, improves comfort, enhances hygiene, and ensures proper product placement, the invention encourages more users to adopt reusable menstrual products. This system not only makes the menstrual care process more accessible and user-friendly but also promotes sustainability by reducing reliance on disposable products. The inclusion of a ergonomic tools, and a hygienic storage holder further enhances the overall user experience, making it a significant advancement in menstrual product technology.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE ACCOMPANYING DRAWINGS

The aforesaid as well as other objects and advantages of the invention will appear hereinafter from the following description taken in connection with the accompanying drawings in which:

FIG. 1 illustrates an exemplary perspective view of the menstrual cup and disc applicator according to an embodiment of the present invention;

FIG. 2 illustrates front and rear view of the menstrual cup and disc applicator according to an embodiment of the present invention;

FIG. 3 illustrates left and right side view of the menstrual cup and disc applicator according to an embodiment of the present invention;

FIG. 4 illustrates top and bottom view of the menstrual cup and disc applicator according to an embodiment of the present invention;

FIG. 5 illustrates the process of insertion of cup in the applicator system according to an embodiment of the present invention;

FIG. 6 illustrates the disc firmly attached to the rod according to an embodiment of the present invention;

FIG. 7 illustrates the process of firm attachment of disc to the hook and notched end of the rod according to an embodiment of the present invention;

### DETAILED DESCRIPTION OF THE INVENTION

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

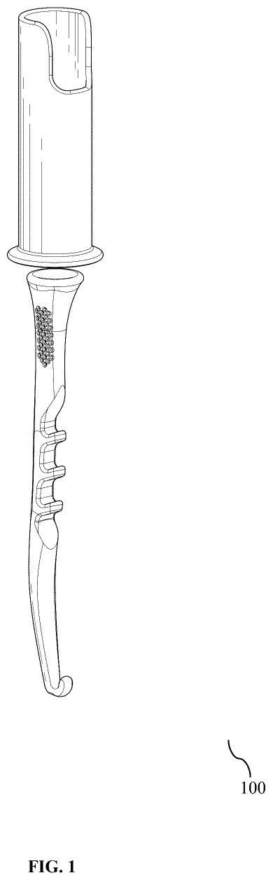

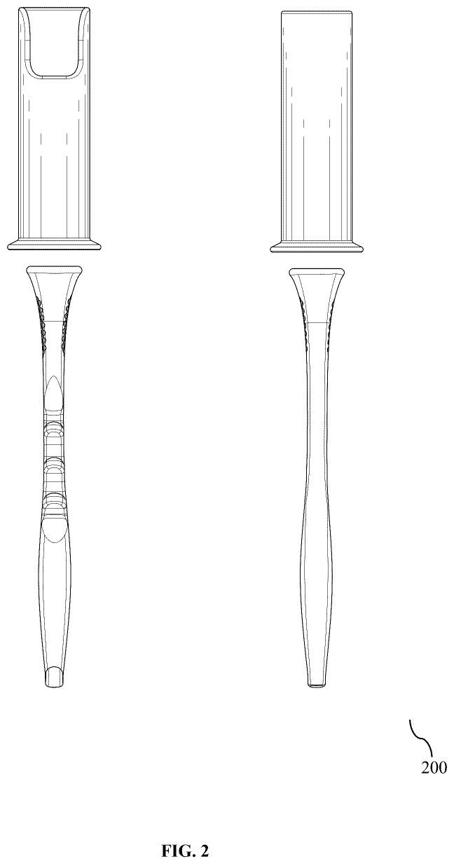

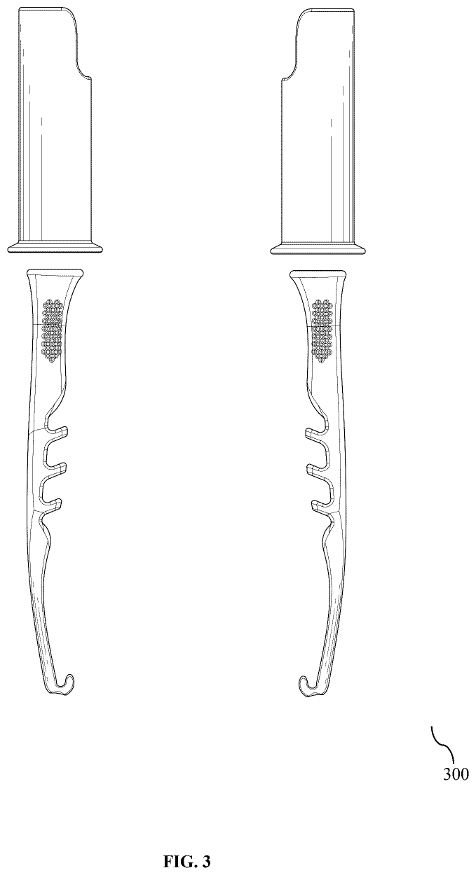

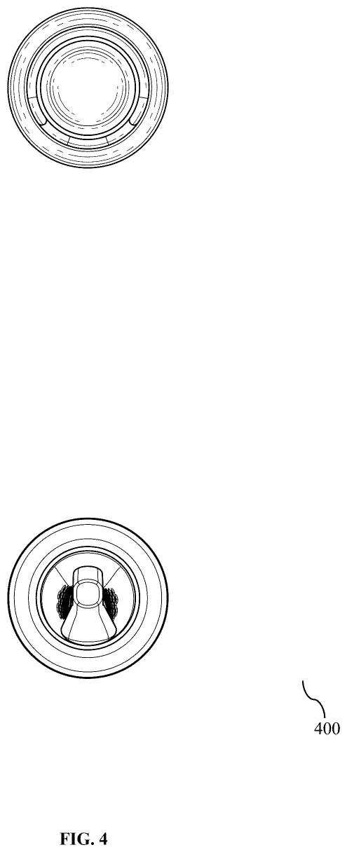

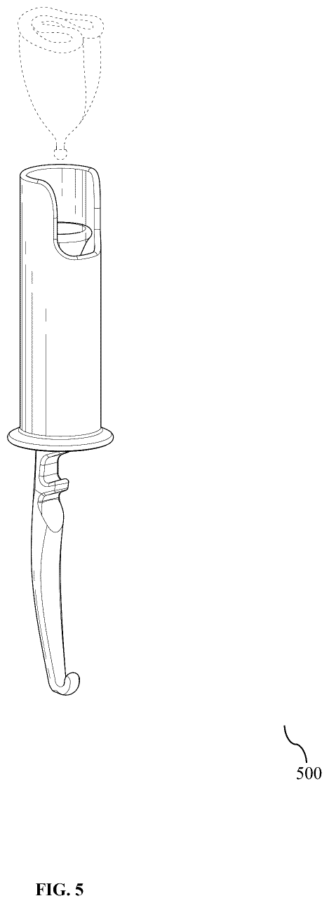

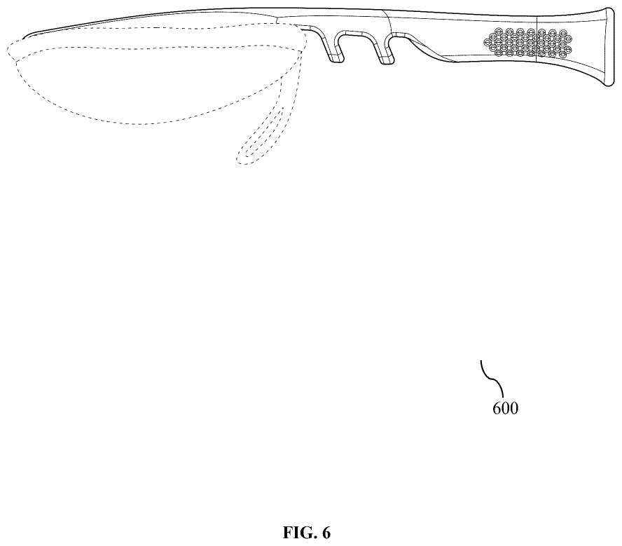

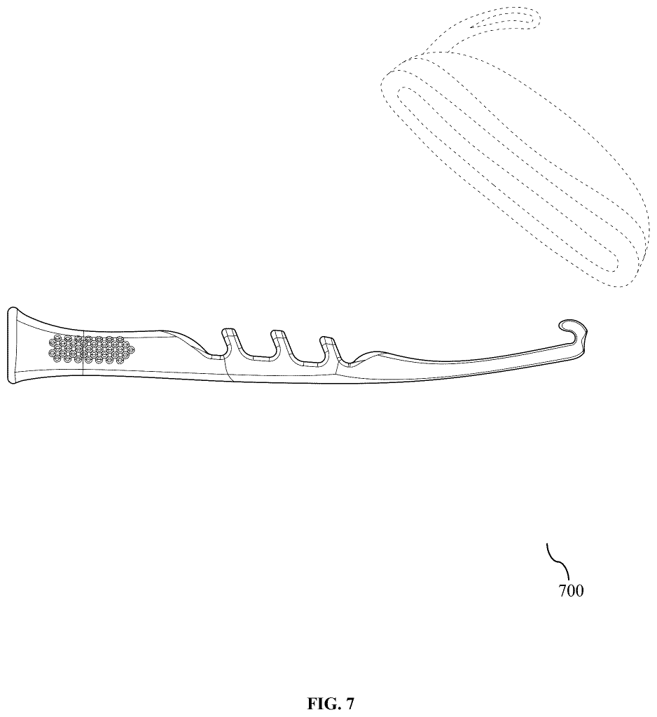

With reference now to the drawings, and in particular from FIG. 1 to FIG. 7 thereof, a multi-functional menstrual product applicator system system for menstrual cups and discs embodying the principles and concepts of the present invention is described.

The cup applicator is the primary tool designed to assist with the insertion of menstrual cups. Its cylindrical body forms the main component and is constructed from medical-grade materials to ensure safety and hygiene. The tube is hollow and smooth on the inside to allow easy sliding of the menstrual cup during the insertion process. At one end of the applicator, there are specialized notches or clamps. These notches are flexible yet sturdy, capable of securely holding various sizes and fold types of menstrual cups (e.g., C-fold or punch-down fold). The applicator's notched end is tapered to facilitate a seamless and comfortable insertion into the vaginal canal, while its smooth contours are designed to reduce the risk of irritation. On the opposite end, an ergonomic grip is provided, allowing the user to maintain control and precision during application. The grip is textured to prevent slipping, even if the user's hands are wet or lubricated.

The cup applicator comes with a detachable insertion rod, which is an essential component for pushing the menstrual cup out of the notched end and into the correct position inside the vaginal canal. This rod is crafted to be lightweight yet strong, ensuring that it can easily slide within the applicator tube without resistance. The rod's tip is rounded to prevent any damage to the cup or discomfort to the user during operation. It is long enough to place the menstrual cup at the appropriate depth, reducing the guesswork for the user. The rod works in perfect synchronization with the notched applicator, delivering the folded menstrual cup gently into position while minimizing friction.

For menstrual disc users, the invention includes a specialized disc inserter and remover rod, a dual-purpose tool designed to address the unique needs of inserting and removing menstrual discs. One end of this rod features small, flexible notches specifically designed to grip the folded disc securely during insertion. These notches hold the disc in place as it is guided into the vaginal canal and release it gently when positioned correctly. On the opposite end of the rod is a smooth, curved hook designed for the removal of menstrual discs. This hook is angled in such a way that it can easily catch the rim or pull-tab of the menstrual disc without causing discomfort or irritation. The rod itself is semi-flexible, allowing it to navigate the natural curvature of the vaginal canal while remaining firm enough for precise control. A textured grip area is located at the midpoint of the rod, ensuring that the user can handle it comfortably and confidently during both insertion and removal processes.

To ensure hygiene and convenience, the invention includes a dedicated storage holder that serves as a compact and portable home for the applicator tools and accessories. The holder is divided into separate compartments that securely house the cup applicator, disc inserter/remover rod, and additional items such as the lubricant bottle. These compartments are precision-molded to prevent shifting or damage to the tools during storage or travel. The holder is made from antimicrobial, medical-grade plastic or silicone, which resists the growth of bacteria and other microorganisms. Its lightweight design and secure locking mechanism make it easy to transport and store safely. Additionally, the storage holder features ventilation holes to allow moisture to escape, ensuring that the tools remain dry and free from mold or odor buildup.

The invention is constructed using high-quality, medical-grade materials to ensure user safety and product durability. The cup applicator and disc rod are made of biocompatible silicone and BPA-free plastic, which are hypoallergenic and free from harmful toxins. These materials are resistant to microbial growth and easy to sterilize, ensuring the highest level of hygiene. The smooth, non-porous surfaces of the tools make them easy to clean and resistant to staining or odor retention. Additionally, antimicrobial coatings may be applied to certain parts of the tools to further enhance their safety and usability.

To help users maximize the benefits of the applicator system, the kit includes a detailed instruction manual. This manual provides step-by-step guidance on using each tool, along with diagrams to illustrate proper techniques. It also includes tips for cleaning and storing the tools, ensuring that users can maintain optimal hygiene and extend the product's lifespan. For first-time users, the manual offers additional advice on how to fold menstrual cups and discs and position them correctly for comfort and effectiveness.

The overall design of the applicator system is focused on ergonomics and user comfort. Each component is carefully shaped to align with the natural anatomy of the vaginal canal, reducing discomfort and ensuring smooth operation. The handles and grips are specifically textured to prevent slipping and improve maneuverability, even in wet or slippery conditions. The applicator and rods are designed to be intuitive to use, making them accessible even for first-time menstrual cup or disc users.

Before using the applicator system, the user must ensure that all components, including the cup applicator, disc inserter/remover rod, and other tools, are thoroughly cleaned and sterilized. The cleaning brush provided with the system can be used to clean the inner surfaces of the applicator and rods, while warm water and mild soap or menstrual product-safe cleaning solutions are recommended. If desired, the user can sterilize the tools in boiling water or a microwave sterilizer, as the materials are made of heat-resistant, medical-grade components. Once cleaned, the components can be dried using a clean cloth or air-dried. Ensuring proper hygiene is crucial to maintaining vaginal health and preventing infections during usage.

For menstrual cup users, the first step involves folding the cup into a smaller shape that can be easily held by the applicator. The user can choose from common folding techniques such as the C-fold, punch-down fold, or 7-fold, depending on their preference and comfort. Once the cup is folded, it is inserted into the notched end of the applicator. The notches are designed to grip the cup securely without damaging its material, ensuring that the fold stays intact during the insertion process. This step allows the cup to fit comfortably within the applicator, ready to be inserted into the vaginal canal.

To ensure a smooth and comfortable insertion, a small amount of the water-based lubricant provided in the system can be applied to the outer edges of the applicator or directly to the folded menstrual cup. The lubricant reduces friction and facilitates easier navigation into the vaginal canal. The hypoallergenic and silicone-safe formulation ensures that the lubricant does not compromise the integrity of the menstrual cup material or cause irritation to sensitive skin. For users who may not need lubricant, this step is optional but recommended for those new to menstrual cups or experiencing dryness.

With the cup securely held within the applicator, the user holds the applicator by the ergonomic grip and gently guides the tapered end into the vaginal canal. The smooth, rounded design of the applicator ensures that insertion is comfortable and minimizes any discomfort. Once the applicator is positioned at the desired depth, the user pushes the insertion rod through the hollow tube of the applicator. The rod gently releases the folded menstrual cup from the notched end, allowing the cup to expand fully and form a seal against the vaginal walls. This ensures a leak-proof fit and proper placement for optimal comfort and effectiveness.

After the menstrual cup is released, the user carefully withdraws the applicator and rod from the vaginal canal. The smooth surfaces and compact design of the applicator make removal easy and comfortable. The user can check the placement of the menstrual cup by lightly rotating or tugging the stem to ensure it has fully opened and formed a seal. The applicator and rod can then be set aside for cleaning or storage. If the cup requires repositioning, the user can perform this manually without needing to reinsert the applicator.

For menstrual disc users, the process begins by folding the disc in half, so it resembles a figure-eight shape or narrow crescent. The folded disc is then placed into the specialized notches at one end of the disc inserter rod. These notches hold the disc securely while the user guides it into the vaginal canal. Using the ergonomic handle for control, the user gently inserts the disc applicator at an upward angle, following the natural curvature of the vaginal canal. Once the disc reaches the vaginal fornix (the area behind the cervix), the user releases the disc by pushing it forward with the insertion rod, allowing it to rest in the correct position.

When it is time to remove the menstrual disc, the curved hook on the opposite end of the disc inserter/remover rod is used. The user inserts the hook end of the rod into the vaginal canal and carefully maneuvers it to catch the rim or pull-tab of the disc. The hook's smooth, rounded design ensures that it gently pulls the disc downward without causing irritation or discomfort. Once the disc is within reach, the user can remove it manually by gripping the rim and pulling it out at a slight angle to avoid spillage.

For first-time users, the system includes an instruction manual with step-by-step illustrations to guide the process. Users are encouraged to take their time during the initial uses to become familiar with the tools and their functionality. Adjustments to the depth of insertion or the angle of the applicator may be necessary to ensure proper placement of the menstrual cup or disc. Over time, users can customize their technique based on comfort and personal anatomy, making the process faster and more intuitive.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims. Additionally, the invention illustratively disclosed herein suitably may be practiced in the absence of any element which is not specifically disclosed herein- and in particular embodiment specifically contemplated, is intended to be practiced in the absence of any element which is not specifically disclosed herein.

1. A menstrual product applicator system for inserting and removing menstrual products, comprising:
a menstrual cup applicator, designed to hold a folded menstrual cup securely in a notched end;
a menstrual disc inserter/remover rod, having one end designed to hold and release a folded menstrual disc and the opposite end comprising a hook for the removal of the disc;
an ergonomic handle for both the cup applicator and disc inserter/remover rod, providing a comfortable grip for the user during insertion and removal.

2. The applicator system of claim 1, wherein the menstrual cup applicator is configured to:
securely hold the folded menstrual cup in a notched end, designed with rounded edges to prevent damage to the cup material during insertion;
release the menstrual cup by a sliding mechanism, which allows for gentle expansion of the cup once it is positioned inside the vaginal canal;
include an ergonomic grip, providing enhanced control and precision during the insertion process.

3. The applicator system of claim 1, wherein the menstrual disc inserter/remover rod includes:
a notched end, designed to hold and release the folded menstrual disc at a specific angle that aligns with the natural curvature of the vaginal canal;
a hook on the opposite end, configured to catch the rim or pull-tab of the menstrual disc during the removal process, with a smooth, rounded tip to prevent discomfort or irritation during removal;
a hollow core, allowing for easy cleaning and sanitation of the rod's internal surfaces.

4. The applicator system of claim 1, wherein the storage holder is designed with:
individual compartments to store the menstrual cup applicator, disc inserter/remover rod, lubricant, and cleaning brush;
antimicrobial properties, preventing the growth of bacteria, mold, or fungi on the tools;
a compact design, allowing for easy transportation and discreet storage of the applicator system.

5. The applicator system of claim 1, wherein the menstrual cup applicator and disc inserter/remover rod are made of:
medical-grade silicone or a similarly flexible, durable material, ensuring safe use and long-term resilience;
materials resistant to high-temperature sterilization, allowing the applicator system components to be safely sterilized in boiling water or a microwave.

6. The applicator system of claim 1, wherein the ergonomic handle of the applicator tools includes:
a non-slip surface, designed to improve user control and minimize slippage during insertion or removal;
a contoured shape, engineered to fit comfortably in the user's hand, reducing strain during use, especially for individuals with limited dexterity or grip strength.

7. The applicator system of claim 1, wherein the the system is designed with features that prioritize user comfort, wherein:
all applicator tools feature smooth, rounded edges, reducing the risk of discomfort or irritation during use;
the applicators and rods are lightweight, ensuring that they do not cause hand fatigue during insertion or removal processes.

8. T The applicator system of claim 1, wherein the menstrual cup applicator can be adjusted to accommodate various sizes of menstrual cups, wherein:
the notched end features a flexible grip, designed to adapt to different cup sizes without losing its hold on the folded cup;
the cup applicator includes interchangeable heads, allowing the user to switch between heads that are sized for small, medium, or large menstrual cups.
